(19) United States
(12) Patent Application Publication
(10) Pub. No.: US 2020/0217849 A1
(43) Pub. Date: Jul. 9, 2020

(54) CAPTURE OF CIRCULATING TUMOR CELLS USING CARBON NANOTUBE SPONGES

(71) Applicants: BingQing Wei, Newark, DE (US); Tong Li, Newark, DE (US); Xin Lucas Lu, Avondale, PA (US)

(72) Inventors: BingQing Wei, Newark, DE (US); Tong Li, Newark, DE (US); Xin Lucas Lu, Avondale, PA (US)

(73) Assignee: University of Delaware, Newark, DE (US)

(21) Appl. No.: 16/624,422

(22) PCT Filed: Jun. 27, 2018
(86) PCT No.: PCT/US2018/039755

§ 371 Date: Dec. 19, 2019

Related U.S. Application Data
(60) Provisional application No. 62/525,942, filed on Jun. 28, 2017.

(51) Int. Cl. G01N 33/574; C12M 1/00; G01N 33/49; A61B 5/055

(52) U.S. Cl. CPC G01N 33/57488; A61B 5/055; G01N 33/491; C12M 47/04

(57) ABSTRACT

The present invention provides a method of capturing circulating tumor cells (CTCs) from a subject without using a cancer biomarker. The method comprises contacting a test sample with a carbon nanotube (CNT) sponge. The test sample comprises cells from a small amount of peripheral blood from a subject after removal of plasma and lysis of erythrocytes. The cells in the test sample comprise CTCs from the subject. The CNT sponge is free of an agent specific for a cancer biomarker.

### CROSS-REFERENCE TO RELATED APPLICATION

This application claims priority to U.S. Provisional Application No. 62/525,942, filed Jun. 28, 2017, the entire disclosure of which is incorporated herein by reference in its entirety for all purposes.

### REFERENCE TO U.S. GOVERNMENT SUPPORT

This invention was made with government support under MURI Grant No. FA9550-12-1-0035 by the U.S. Air Force Office of Scientific Research and NIGMS-IDeA Grant No. U54-GM104941 awarded by the U.S. National Institutes of Health. The United States has certain rights in the invention.

### FIELD OF THE INVENTION

The invention relates generally to the use of a marker-free carbon nanotube (CNT) sponge to capturing circulating tumor cells (CTCs) from a subject.

### BACKGROUND OF THE INVENTION

Recent advances in nanomaterials have shed new light in biomedical applications, such as drug delivery, disease diagnosis, and tissue engineering. In animal tissues and organs, collagen fiber networks compose extracellular environments, in which cells can function and progress. Various 3-dimensional carbon-nanotube sponges (CNT sponges) have recently been developed and characterized. These nano-materials share similar structures and mechanical properties with collagen networks in biological tissues.

A majority of tumor patients die of metastatic diseases (90% of tumor-related mortalities). Metastasis usually involves the following stages: local invasion of tumor cells (e.g., lymph nodes), circulation through blood, reallocation in distant tissue/organ environment, and formation of secondary tumors. Before spreading into healthy tissues, circulating tumor cells (CTCs) may have been accumulated in patients' blood for months or years. Only after secondary tumors are formed in patients are these metastatic lesions detected by imaging techniques. While the patients may then have become symptomatic, most treatments would not be effective. Early application of chemotherapies on the CTCs-positive/metastatic-lesions-negative patients may increase effectiveness of chemotherapies and improve prognosis. In addition, several aggressive cancer types, for example, triple negative breast cancer, require immediate commencement of chemotherapies, which may result in over-treatment. Successful elimination of CTCs as evidenced by liquid biopsy may enable physicians to improve therapeutic treatment plans.

To date, CTC isolation methods are mainly divided into two major categories, microfluidic devices and immunomagnetic selection devices, which employ physical properties (e.g., size and density gradient) and protein expression of the CTCs, respectively. The physical-properties-based techniques face the problem of accuracy due to the overlapping physical properties between CTCs and leukocytes. The immunomagnetic technique relies on specific protein expression of tumor cells (e.g., EpCAM), which is not positive for all types of CTCs. For example, at least 7.5 ml of blood from a subject is commonly required in the operation of immunomagnetic techniques.

With the development of nanomaterials, several nano-engineered devices have been developed to capture CTCs by taking advantages of highly adhesive properties of tumor cells on specific nanomaterial surfaces. These devices include nano-pillars, nanowires, and nano-fibers. Carbon nanomaterials such as carbon nanotubes and graphene have also been employed to assist in the capture of CTCs. However, similar to immunomagnetic selection methods, these techniques require additional tumor-specific adhesive molecules such as proteins for specific recognition, which both increases operation costs and limits detectable cancer types. CTC-iChip was developed to isolate CTCs from blood by negative depletion of leukocytes based on leukocyte-specific markers.

There remains a need for an effective and efficient method of isolating CTCs from a small amount of blood from a subject without using any tumor biomarkers.

### SUMMARY OF THE INVENTION

This invention relates to the use of a carbon nanotube (CNT) sponge to capture circulating tumor cells (CTCs) from a subject and the uses of the captured CTCs.

A method of capturing circulating tumor cells (CTCs) from a subject without using a cancer biomarker is provided. The method comprises contacting a test sample with a carbon nanotube (CNT) sponge for no more than 60 minutes. The test sample comprises cells from no more than 2 ml peripheral blood from a subject after removal of plasma and lysis of erythrocytes. The cells in the test sample comprise CTCs from the subject. The CNT sponge is free of an agent specific for a cancer biomarker. Thus, the CTCs are captured by the CNT sponge.

The CTCs may not have the cancer biomarker. The cancer biomarker may be specific to tumor cells. The subject may have the tumor. The tumor may be selected from the group consisting of breast cancer, lung cancer and colorectal cancer. The cancer biomarker may be selected from the group consisting of EpCAM, cytokeratins, CD45 and HER2. The test sample may comprise 200-1,000 CTCs per ml of the peripheral blood from the subject.

The method may comprise contacting the test sample with the CNT sponge for no more than 30 minutes. The method may comprise contacting the test sample with the CNT sponge for no more than 15 minutes.

At least 20% of the CTCs in the test sample may be captured by the CNT sponge. At least one of the captured CTCs may remain viable after 7 days in a cell culture.

The method may further comprise detaching the captured CTCs from the CNT sponge. The method may further comprise incubating the captured CTCs in a culture medium. The method may further comprise characterizing the captured CTCs.

### BRIEF DESCRIPTION OF THE DRAWINGS

FIG. 1 shows preparation of CTC capture chip and the CTC isolation strategy based on 3D CNT sponge. (a) The mechanical and structural similarity between CNT sponge and nature soft tissues. (b) Similar microstructures between CNT sponge and collagen fiber networks in cartilage. (c) Method design of the capture process. 3D CNT sponge is fabricated by chemical vapor deposition (CVD) method, and the CNT sponge is embedded in glass slides for CTC capture purpose. (d) The cell suspension is placed on a CNT sponge surface and cultured for 15-60 minutes so that CTCs can attach to the CNT structure. The sponge is then flipped for the unbound cells to be released into the culture medium by gravity.

FIG. 2 shows CTC capture efficiency. (a) Breast cancer CTCs captured in 60 minutes (b) Lung cancer CTCs captured in 60 minutes. For (a) and (b), all scale bars equal 100 μm. (n=3, * P<0.002, ** P<0.001, *** P<0.0001). (c) The immunofluorescence imaging of CTCs captured by the CNT sponge. Both breast cancer cells and lung cancer cells were tested. Positive biomarkers of CK and EpCAM confirm that all captured cells are CTCs. The scale bars in all sub-figures represent 100 μm.

FIG. 3 shows CTC clinical validation. (a) The protocol of CTC capture by the CNT sponge from clinical samples. (b) and (c) shows the cells captured by CNT sponge from two different clinical samples. EpCAM was marked. In both immunofluorescence images, the nuclei of the cells are stained with DAPI (round grey dot). The scale bars represent 100 μm. (d) The CTC counts from these two clinical samples.

FIG. 4 shows SEM characterization of CTCs. (a) Cell morphologies of breast cancer cells (cell line: MDA-MB-231) and lung cancer cells (cell line: NCI-H322) on the CNT sponges and glass-f, more rounded morphology are found on the surface of the CNT sponge, while most of the cells are of elongated morphology on the glass surface. (b) Detailed morphological information of the CTCs accommodated on the CNT sponges (top) and flat glasses (bottom).

FIG. 5 shows statistical results of cell movement on CNT sponge. (a) Typical moving tracks of breast cancer cells on CNT sponge and glass slides and the cell motion is also summarized (n=4, F<0.001). (b) The residue breast cancer cells (F-actin stained by Phalloidin 488) on the CNT sponge and glass coated with fibronectin after long trypsinization (0.25%, 10 minutes). (c) The syntheses of collagen and glycoproteins were both significantly promoted in the CTCs on the CNT sponge comparing to glass coated with fibronectin in 24-hour. The newly synthesized collagen and glycoprotein were fluorescently dyed with MB-488. (d) Fluorescence intensity represents the amount of ECM synthesized by the breast cancer cells. (n=3 and *** P<0.0001). All scale bars represent 50 μm.

### DETAILED DESCRIPTION OF THE INVENTION

The present invention relates to an effective and efficient method of capturing circulating tumor cells (CTCs) from a subject by a carbon nanotube (CNT) sponge without using biomarkers. The invention is based on a surprising discovery of an outstanding binding affinity between CTCs and CNT sponges due to both invasive characteristics of tumor cells and structural/mechanical similarity of the CNT sponges to the natural extracellular matrix (ECM). Validated by both tumor-cell-spiked blood samples and clinical samples, sparse CTCs can be effectively and efficiently captured by CNT sponges. The captured CTCs may remain vital on the CNT sponges, enabling further characterization of the CTCs, for example, pathological analysis. As a low cost and simple operation technique, this CNT-sponge-based capture method provides biomarker-free CTC capture platforms for early detection of metastatic disease using a liquid biopsy. The CNT sponges serve as an ideal artificial niche for eukaryotic cells to attach and proliferate, earning new opportunities of the CNT material in biomedical applications.

The term “circulating tumor cells (CTCs)” used herein refers to cells in vasculature or lymphotics that are derived from a primary tumor in a subject and are carried around the body of the subject in circulation. The CTCs may be accumulated in the blood of a subject having a tumor, and may be present at 1-10 CTCs per mL of whole blood of patients with a tumor, for example, cancer. Isolation of CTCs from the blood of a patient having a tumor may be considered a liquid biopsy from the patient, providing live information about the patient, for example, metastasis status, disease progression and treatment effectiveness. The CTCs are CD45 negative, indicating that these cells are not of hematopoietic origin, and may or may not have a cancer biomarker.

The CTCs may be traditional CTCs, cytokeratin negative CTCs, apoptotic CTCs or small CTCs. The traditional CTCs are confirmed cancer cells with an intact, viable nucleus and express cytokeratin and cancer biomarkers. The cytokeratin negative (CK⁻) CTCs are cancer stem cells or cells undergoing epithelial-mesenchymal transition, and express cancer biomarkers, but not cytokeratin. Apoptotic CTCs are traditional CTCs that are undergoing apoptosis and show nuclear fragmentation or cytoplasmic blebbing associated with apoptosis. The change in the ratio of traditional CTC to apoptotic CTCs in a patient under a therapy provides evidence of the therapy's efficacy in targeting and killing cancer cells. Small CTCs have sizes and shapes similar to white blood cells, but express cytokeratin and cancer biomarkers. Small CTCs have been implicated in progressive diseases and differentiation into small cell carcinomas, which often requires a different therapeutic course.

The term “biomarker” used herein refers to a measurable indicator of some biological state or condition, for example, a special protein on cancer cells. The biomarker may be a substance whose presence in an organism indicates a phenomenon in the organism such as a disease, condition or environmental exposure. The biomarker may be a chemical compound, a biological molecule (e.g., a protein, a nucleic acid or a liquid), or a combination thereof. The biomarker may be present in a cell, for example, in or on a cell, indicating the origin or specific property of the cell. Biomarkers are widely used for diagnosis, treatment or isolation of cells having the same biomarkers.

The terms “cancer biomarker” used herein refers to a biomarker associated specifically with a tumor or cells of a tumor. A tumor is a mass formed by an abnormal growth of cells. The tumor may be benign, pre-malignant or malignant. A malignant tumor is also called cancer. A cancer biomarker may be present in or on tumor cells, which are cells derived from a tumor, for example, a cancer. Exemplary cancer biomarkers include EpCAM, cytokeratins, CD45, HER2 and Maspin.

The term “agent specific for a cancer biomarker” used herein refers to a substance that binds specifically to a cancer biomarker. The agent specific for a cancer biomarker may be a chemical compound, a biological molecule (e.g., a protein, a nucleic acid or a liquid), or a combination thereof. In one embodiment, the agent specific for a cancer biomarker is an affinity-binding molecule such as an antibody or a fragment thereof that binds specifically to the cancer biomarker. Examples of agent specific for a cancer biomarker include anti-EPCAM antibody.

The term “carbon nanotube (CNT) sponge” used herein refers to an allotrope of carbon with a porous cylindrical nanostructure. The CNT suitable for the present invention has an outstanding binding affinity with CTCs as cells tend to grow dendrites surrounding the CNTs. The CNT may or may not have an agent specific for a cancer biomarker. The binding between the CNT sponge and the CTCs according to the present invention is independent from any biomarker or an agent specific for a cancer biomarker. In one embodiment, the CNT sponge is free of antibodies or proteins.

The term “captured” used herein refers to certain cells being separated from other cells in a sample by binding to a CNT sponge. At least about 1%, 5%, 10%, 15%, 20%, 25%, 30%, 40%, 50%, 60%, 70%, 80%, 90% or 95% of the CTCs may be captured by the CNT sponge. At least about 1%, 5%, 10%, 15%, 20%, 25%, 30%, 40%, 50%, 60%, 70%, 80%, 90% or 95% of the cells captured by the CNT sponge are CTCs.

The term “subject” used herein refers to an animal, preferably a mammal. The mammal may be a human. The subject may be a patient having a tumor, for example, a cancer. Exemplary tumors include cystosarcoma, islet cell carcinoma, hepatoma and malignant carcinoid. Exemplary cancers include breast cancer, lung cancer, colorectal cancer and pancreatic cancer. In one embodiment, the subject is a patient has a tumor, for example, a cancer.

A method of capturing circulating tumor cells (CTCs) from a subject without using a cancer biomarker is provided. The method comprises contacting a test sample with a carbon nanotube (CNT) sponge for no more than about 5, 10, 15, 30, 45, 50, 60, 90, 120 or 180 minutes. The test sample comprises cells from no more than 0.5, 1.0, 1.5, 2.0, 2.5, 3.0, 3.5, 4.0, 4.5, 5.0, 5.5, 6.0, 6.5 or 7.0 ml peripheral blood from a subject after removal of plasma and lysis of erythrocytes. The cells in the test sample comprise CTCs from the subject. The CNT sponge is free of an agent specific for a cancer biomarker. As a result, the CTCs are captured by the CNT sponge without using a cancer biomarker.

The CTCs may not have a cancer biomarker. The cancer biomarker may be specific to tumor cells, i.e., present in tumor cells, but not non-tumor cells. The tumor may be a breast cancer, lung cancer or colorectal cancer. The cancer biomarker may be EpCAM, cytokeratins, CD45, HER2 or a combination thereof.

The test sample may comprise about 1-100,000, 0-10,000, 1-1,000, 1-500, 1-200, 1-100, 1-50, 1-10 or 1-5 CTCs from the subject. In one embodiment, the test sample comprises 1-10 CTCs from the subject.

According to the present invention, at least about 1%, 5%, 10%, 15%, 20%, 25%, 30%, 40%, 50%, 60%, 70%, 80%, 90% or 95% of the CTCs in the test sample are captured by the CNT sponge. In one embodiment, at least 25% of the CTCs are captured.

According to the present invention, at least about 1, 5, 10 or 50 of the captured CTCs may remain viable after about 1, 2, 3, 4, 5, 6, 7, 10 or 14 days in a cell culture. In one embodiment, at least one of the captured CTCs remain viable after 7 days in a cell culture. At least about 10%, 20%, 30%, 40%, 50%, 60%, 70%, 80%, 90% or 95% of the captured CTCs may remain viable after about 1, 2, 3, 4, 5, 6, 7, 10 or 14 days in a cell culture.

The method may further comprise detaching the captured CTCs from the CNT sponge. The sponge may be flipped over so that cells not attached on the CNT sponge are released from the sponge by gravity. The CTCs captured by the CNT sponge may be detached by cell lifting chemicals, for example, trypsin or EDTA. Detached CTCs can be cultured in Petri dish with culture medium at a temperature of about 15-40° C., 20-37° C., 25-37° C., or about 25° C. or 37° C., for at least about 0.5, 1, 2, 3, 6, 12, 18 or 24 hours. At least about 10%, 20%, 30%, 40%, 50%, 60%, 70%, 80%, 90% or 95% of the captured CTCs may remain alive. At least about 1, 5, 10 or 50 of the captured CTCs may remain alive.

The method may further comprise incubating the captured CTCs in a culture medium. The culture medium may be minimum essential medium, serum, antibiotics or a combination thereof. The captured CTC may be cultured at a temperature of about 15-40° C., 20-37° C., 25-37° C., or about 25° C. or 37° C. At least about 10%, 20%, 30%, 40%, 50%, 60%, 70%, 80%, 90% or 95% of the captured CTCs may remain viable after 1, 2, 3, 4, 5, 6, 7, 10 or 14 days in a cell culture. At least about 1, 5, 10 or 50 of the captured CTCs may remain viable after about 1, 2, 3, 4, 5, 6, 7, 10 or 14 days in a cell culture. In one embodiment, at least one of the captured CTCs remain viable after 7 days in a cell culture.

The method may further comprise characterizing the captured CTCs. For example, the CTCs may be labeled with an agent specific for a cancer biomarker to detect the presence of the cancer biomarker in or on the captured CTCs. The synthesis of extracellular matrix molecules such as collagen and glycoprotein by the captured CTCs may be detected and analyzed. The characterization of the captured CTCs from a patient may be used to determine metastasis status, disease progression and treatment effectiveness in the patient.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Efficient Capture of Circulating Tumor Cells Using Marker-Free Carbon Nanotube Sponges

The identification of circulating tumor cells (CTCs) in peripheral blood is significant to decide the risk of metastasis in cancer patients. In order to capture the CTCs from a small amount of peripheral blood, a new CTC capture chip is designed based on 3D carbon nanotube (CNT) sponges. Without tumor-specific molecules, this CTC capture chip can efficiently capture CTCs from 1 ml peripheral blood in 60 minutes. The captured CTCs can be detached from the CNT sponge and remains vital in long-term in vitro cell culture. The captured CTCs demonstrate high binding affinity and aggressive synthesis of extracellular matrix (ECM) on the CNT sponges. This 3D CNT-sponge-based CTC capture chip is an ideal candidate for the next-generation, marker-free CTC isolation devices.

### Material and Methods

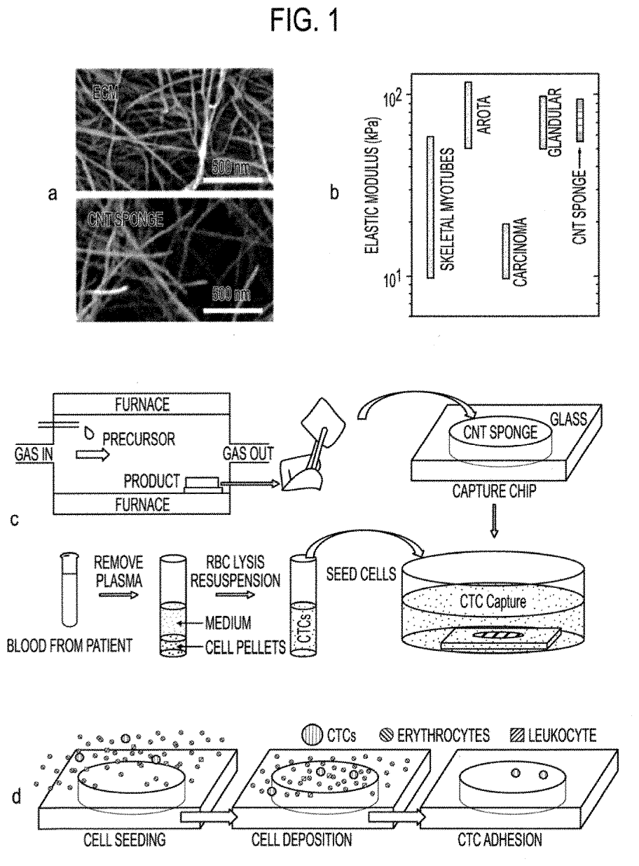

Inspired by the natural structure of ECM, which is a crucial component of the metastatic niche and holds an outstanding binding affinity to CTCs, the 3D CNT sponge is employed to mimic the tumor cell niche and capture CTCs from the peripheral blood. This consideration is based on the similar structural and mechanical behavior of the naturally ECM-based connective tissues (FIG. 1a). The process of this CTC isolation technique is illustrated in FIG. 1b, including the fabrication of CTC capture chip and the process of CTC capture. The 3D CNT sponge was fabricated by chemical vapor deposition (CVD) method, and then the CNT sponge was embedded into glass slides to perform as the capture chip. Besides clinical samples, tumor cells at various densities were spiked into a fresh human blood sample after removing plasma. The cells were given 15 to 60 minutes to attach to the chip surface. Afterwards, the chips were flipped so that the unattached cells would be released back into the medium by gravity (FIG. 1c).

CNT Sponge Fabrication

CNT sponges were synthesized by chemical vapor deposition (CVD) process using ferrocene and 1,2-dichlorobenzene as the catalyst precursor and carbon source. Ferrocene powders were dissolved in dichlorobenzene, which was then continuously injected into a 2-inch quartz tube in a resistive furnace by a syringe pump (0.13 ml/min). The reaction temperature was 860° C. A mixture of Ar and H₂ flows at a rate of 2000 ml/min and 300 ml/min, respectively. A quartz sheet was placed in the reaction zone as the growth substrate. Sponge-like products were collected from the quartz substrate after the CVD process.

CNT sponges were treated in 5% HCl for 3 days to remove the catalysts and kept in DI water. Ahead of using, the sponges were sterilized in an autoclave to reduce the risk of contamination in cell culture. From a large piece of CNT sponge, cylindrical samples with a diameter of 1.5 mm were punched using a biopsy punch and embedded into a piece of glass with pre-drilled holes for easy operation in following experiments.

Cell Culture

Tissue culture reagents were purchased from Sigma-Aldrich (St. Louis, Mo.) unless otherwise specified. The MDA-MB-231 cell line was purchased from Sigma-Aldrich and cultured in L-15 Medium (Leibovitz) supplemented with 15% fetal bovine serum, 1% penicillin-streptomycin, and 2 mM L-Glutamine solution. NCI-H322 cell was purchased from Sigma-Aldrich and cultured in RPMI-1640 Medium supplemented with 10% fetal bovine serum, 1% penicillin-streptomycin, and 2 mM L-Glutamine solution.

For 2D cell culture in a Petri dish, sub-passage was carried out with 0.25% trypsin (Gibco) when the Petri dish is 80% full, and the seeding density was maintained at ˜3×10⁴ cells/cm². For suspension culture, the density of cells was prepared according to experimental design (e.g. 10⁵ cells/ml). The cell suspension medium was transferred into a spinner flask (ChemGlass, CLS-1410-25), which was placed on a magnetic stirrer (IKA, MINI MR) with a rotation speed of 60 rpm in the cell culture incubator (ThermoFisher). Cells after sub-passage can fully recover in 24 hours.

Patient Blood Samples

Patient blood samples were obtained according to an approved protocol under the supervision of the institutional review board of Christiana Care Health System, Newark, Del., U.S. The informed signed consent was obtained from either the patient at the time of sample collection. Samples were de-identified and all protected health information and patient identifiers were removed. The blood samples are processed right after the delivery, including plasma removal and RBC lysis. The residue blood contents (ideally including leukocytes and possible CTCs) was dropped to a 1.5 ml conical vial that is embedded by CNT sponge (performs as a CTC capture chip). After 60 minutes, the surface of CNT sponge was gently washed to remove the unattached cells.

Tumor-Cell-Spiked Blood Samples

Permission of using de-identified human blood samples was obtained from the University of Delaware, Newark, Del., U.S. De-identified fresh human blood sample was purchased (Zen-Bio, U.S.). The plasma was removed by centrifuging the blood at 2000 g for 10 minutes. Cancer cells (either MDA-MB-231 and NCI-H322) were then spiked into the plasma-free cell suspension (RPMI 1640, Sigma-Aldrich) with designed concentrations.

Cell Labeling

To measure the cancer cell mobility, live cell tracker (ThermoFisher CellTracker™ Red CMTPX) was used to stain the cells after full attachment on the designed material surface (4-6 hours for glass surface and 1 hour for the CNT sponge surface). To study the cell morphology using immunofluorescence imaging, F-actin in cytoskeleton was stained by Phalloidin (Santa Cruz, CruzFluor™ 488 Conjugate), and the nuclei were stained with DAPI (Santa Cruz).

For the immunofluorescent labeling of cancer cells, Mouse Anti-Human Cytokeratin 8 Monoclonal Antibody and the Anti-Mouse IgG Secondary Antibody (R&D system) were used to treat the formaldehyde-fixed cells. A similar protocol was adopted in identifying EpCAM by using the primary antibody of Mouse Anti-Human EpCAM/TROP-1 Monoclonal Antibody (R&D system). Both cytokeratin and EpCAM are unique markers on cancer cells. To identify white blood cells, Mouse Anti-Human CD45 Monoclonal Antibody was used. Selection of the secondary antibody depends on the color combination in the final image, and commonly emission wavelength of 658 nm (red) and 574 nm (green) were adopted for the separation purpose while co-using with DAPI (nuclei staining in blue).

Time-Lapse Imaging of Cell Mobility

After seeding on the designed material surface for 24 hours, the MDA-MB-231 cells were stained with live cell tracker (CMTPX, ThermoFisher) as aforementioned. Zeiss LSM 510 Meta Confocal Microscope imaging system was used to record the motion of living cells. In time-lapse mode, a fluorescent image of cells was recorded every two minutes for 60 minutes. After obtaining the time-lapse images, TrackMate in ImageJ (1) was used to allocate the cells and track the motion of cells.

Cell Capture Experiment

MDA-MB-231 and NCI-H322 cells were prepared in the medium at a density of 10⁴ cells/ml and undergone suspension culture as aforementioned. After 24-hour culture, the cell suspension medium was carefully injected into a 12-well cell culture plate (Corning) with CNT sponge samples or flat glass slides (with fibronectin coating). Each culture well was filled with 2 ml cell suspension medium, which distributed ˜100 cells per 1 mm² area. The time-points of 15, 30, 45, and 60 mins were chosen to test the temporal sensitivity of the CNT sponge and fibronectin-coated glass slide. After each time point, the samples were flipped, so that the unattached cells will be fallen back into the suspension medium from the material surface by gravity. Afterward, the samples were transferred into a fresh medium and cultured for another 24 hours before counting on Zeiss LSM 510 Meta Confocal Microscope imaging system. For ensuring the accuracy of cell counting, DAPI was used to stain the nuclei of the CTCs. The imaging area is 0.49 mm² (700 μm×700 μm) by using a 10× objective. Each experiment was repeated with three different samples for statistical reliability purpose.

Cell Viability Study

CNT sponges or glass slides with captured CTCs were cultured in the medium for up to one week. On 1, 3, and 7 days, the viability of the CTCs was examined. The cell-CNT sponge samples were stained by incubation with 1.0 mL of PBS containing 0.4 μL calcein-AM per 13 μL EthD-1 (ThermoFisher, Live/Dead Viability/Cytotoxicity Kit [L-3224]) for 30 minutes at room temperature. Zeiss LSM 510 Meta Confocal Microscope imaging system was used to obtain an image of live and dead cells.

SEM Characterization of Cell Morphology

After culturing for a designed time, cells were fixed in 4% Formaldehyde and 3% Glutaraldehyde solution for overnight. After fixation, an Ethanol solution (in DI water) with a concentration of 25, 50, 75, and 100% was sequentially applied on samples for 15 minutes each time, and the final 100% Ethanol treatment was repeated for one more time. Afterward, hexamethyldisilazane (Alfa Aesar) in 100% Ethanol with the concentrations of 25, 50, 75, and 100% were gradually applied to the samples, while the 100% hexamethyldisilazane treatment was also repeated. Samples were dried in the chemical hood for overnight. After metal sputtering, cells on the material surface were examined using a JEOL JSM-7400F field emission scanning electron microscope operated at 3 kV and 10 μA.

Mechanical Testing of CNT Stiffness (in Cell Culture Medium)

Mechanical stiffness of the 3D CNT sponge was measured by an unconfined compression test. The original thickness of each cylindrical 3D CNT sponge sample was measured as the distance between the upper and lower loading platens with a 5-g tare load. During the test, a 20% strain was applied to the CNT sponge at a constant speed of 2 μm/s. After reaction force had reached an equilibrium state, Young's modulus of the sample was determined from the recorded force. The mechanical testing was performed in the cell culture medium (RMPI 1640 medium+10% FBS+2 mM L-Glutamine) to measure the sample stiffness perceived by CTCs.

Characterization of GAG and Collagen Synthesis

The newly synthesized GAG or collagen molecules were fluorescently marked using a bioorthogonal chemistry technique. After 24-hour recovery after sub-passage, the CTC samples (on glasses and CNT sponge) were fluorescently labeled using the click chemistry technique. Unnatural amino acid L-Azidohomoalanine (AHA, ThermoFisher), and N-azidoacetyl galactosamine-tetraacylated (Ac4GalNAz-GAL, ThermoFisher) were used in this experiments. MB488 was used in the click reaction. Labeling result images after 24-hour culture were obtained by using Zeiss LSM 510 Meta Confocal Microscope.

Fluorescence Intensity Measurement

After fluorescent imaging, the samples were digested by Papain (ThermoFisher), so that the amount of new GAG or collagen contents can be tracked by reading the fluorescent intensity of the digestion solution. The concentration of ECM fragments in the conditioned medium was measured by reading the fluorescent intensity (at 515 nm) of solution using a microplate reader (Gemini EM, Molecular Devices).

Statistics Analysis:

One-way ANOVA was performed to compare the results from two different groups, and the corresponding significance levels (P value) were marked out in each image. For the comparison among three groups, the strategy of F-test was adopted and the significance levels (F Value) was provided in the statistical results.

Results and Discussion

Two common tumor cell lines with distinct genotypes, breast cancer cells (MDA-MB-231) and lung cancer cells (NCI-H322), were chosen as the capture targets in vitro. Our capture strategy was designed to be non-selective to the types of cancers, which is independent of the unique immunochemical characteristics of CTCs. Therefore, no biomarkers or ligands were used to treat the 3D CNT sponges. The capture efficiency, accuracy, and phenotype of captured cells were studied using both cancer cell lines. Peripheral blood samples from triple negative breast cancer (TNBC) patients were collected and tested to validate the feasibility of this method in the clinical application.

CTC Capture Efficiency

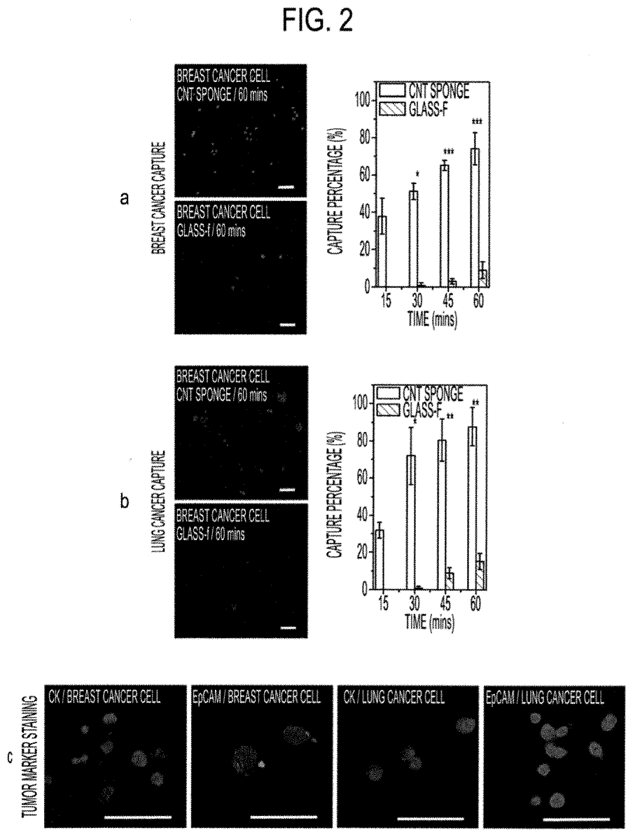

At first, a cell suspension medium at a density of 10⁵ cells/nil of either breast cancer cells or lung cancer cells was prepared and cultured in suspension using a spinner flask for 24 hours. This high concentration does not correspond to the physiological condition in patients and was only designed to study the capture efficiency, where large CTC number can promise higher statistical reliability. The cell suspension medium was then dropped on the sponge and left on the capture chip for 15, 30, 45, or 60 minutes. Afterward, the sponge was flipped and washed gently with a cell culture medium. In order to distinguish the CTC capture capability of the CNT sponges, glass substrates coated with fibronectin were tested as the control group (referred as glass-f). To visualize and count the captured cells, the cell nucleus was stained with DAPI (blue). Fluorescent images of the captured cells were taken on a laser confocal microscope (10× objective, Zeiss LSM 510) and presented in FIGS. 2a and 2b. The 3D CNT sponges are sensitive to both breast cancer cells (MDA-MB-231) and lung cancer cells (NCI-H322), and a significant amount of CTCs was captured by the sponges in merely 15 minutes, while no CTCs can be found on the glass-f slides. The capture percentage (p_{c}) is calculated by dividing the number of the captured CTCs (n_{c}) by the seeding number of cells per image-scope (nₜ). In 30 minutes, CNT sponges can capture 50-70% of the CTCs from the suspension medium, while glass-f slides still barely capture any CTCs. A limited number of CTCs can be observed on glass-f slides when the seeding time is 45 minutes or longer. For both types of tumor cells and all seeding time, the CNT sponges captured significantly more CTCs than the glass-f slides (n=3, P<0.001). More fluorescent images about the

A de-identified fresh human blood sample was purchased (ZenBio, U.S.), healthy adult (52 years, Male, Hispanic). After plasma removal and erythrocytes lysis, the leucocytes pellet was diluted in a cell culture medium, as illustrated in FIG. 1b. Tumor cells (MDA-MB-231 or NCI-H322) were spiked into the blood cell suspension at a density of 10³ cells/ml that is within the physiological level. The medium with mixed cells was dropped on the CNT capture chip followed by 60 minutes of seeding time. Following an established staining protocol, the captured cells were stained for both cytokeratin (CK) and epithelial cell adhesion molecule (EpCAM) expressions, which indicate the malignancy. DAPI was used to represent the nuclei, while CK and EpCAM were marked with IgG conjugated to fluorescent agents. On almost all captured cells, both CK and EpCAM can be found (FIG. 2c), proving that they are tumor cells instead of blood cells.

The CNT chips with captured cells were cultured in the lab for an extra 7 days, and then the cell viability was examined by Live/Dead staining kits. The majority of the captured CTCs (˜80%) remained vital after 7 days, which allows further immunocytochemical studies on the captured CTCs according to specific clinical or research requirements.

Clinical Samples Validation

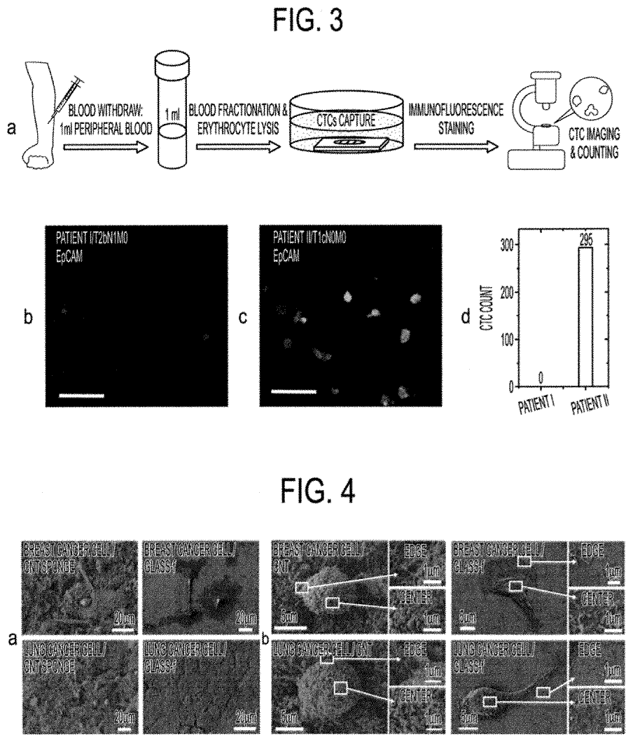

Clinical samples were also tested to validate the feasibility of this CTC capture chip in clinical applications. Patient samples were obtained under informed consent under the supervision of the institutional review board at Christiana Care Health System, Newark, Del., U.S. Two blood samples from TNBC patients were employed. The pathology report of Patient I (T2BN1M0) shows that regional lymph nodes that are involved, while Patient II (T1CN0M0) has a smaller tumor size and no lymph nodes were affected. The detailed procedure of CTC capture from a clinical sample is provided in FIG. 3a. After erythrocyte lysis, the cells (including leukocytes and possible CTCs) were allowed to attach on the CTC capture chip for 60 minutes. The EpCAM and nucleus were individually stained and the immunofluorescence images are provided in FIG. 3b-c. It can be found that a number of CTCs (295 EpCAM positive cells, FIG. 3d) can be captured from Patient I while no CTCs can be found in the blood of Patient II, which agrees with the conclusion of pathology reports.

Biophysics of the CTC Capture by 3D CNT Sponge

Both CNT sponges and glass-f slides with tumor cells were treated for SEM characterization (FIG. 4a). Morphologies of the attached tumor cells can be mainly divided into two categories: elongated and rounded shapes. Both breast cancer cells and lung cancer cells on glass-f slides stabilized at the elongated morphology, while most tumor cells end up with the rounded morphology of the CNT sponges. The elongated and rounded morphologies correspond to different tumor invasion modes, mesenchymal-type movement, and amoeboid movement, respectively. The rounded shape or amoeboid movement of CTCs indicates a high chance of tumor arising from connective tissue cells, leading to a high rate of metastasis, which is consistent with our primary hypothesis that CNT sponge provides a mock connective tissue environment for the CTCs to progress in. The detailed interaction between CTC edges and substrate materials (CNT sponge and glass-f) are provided in FIG. 4b. For both breast cancer cells and lung cancer cells, the edges of cell body or plasma membrane embraced multiple CNTs, while no such interactions can be achieved on the glass slides coated with fibronectin. In comparison to healthy cells, the invasiveness of tumor cells is an essential characteristic for metastasis. There are significant cleavages on the CNT sponge near the border of the rounded tumor cells (FIG. 4b, marked in grey around the cells), which reflects the strong interaction between tumor cells and the CNT sponge. In contrast, on the glass-f slides, the spreading of cells is mainly achieved by the remodeling of F-actin networks and the cell moves by the polarized protrusion of F-actin, which is different from the aggressive phenotypes of CTCs (rounded shape) on the CNT sponges.

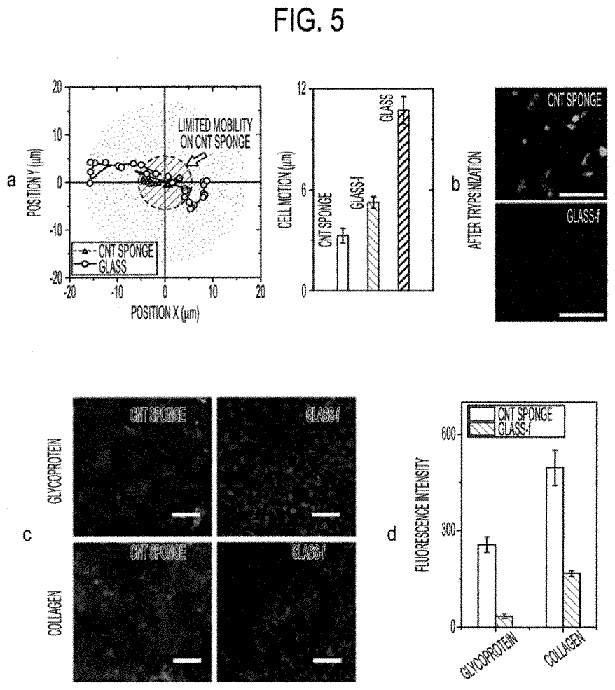

The mobility of CTCs was studied to evaluate the binding affinity between cells and the CNT sponge, in which live cell tracking technique is combined with the time-lapse laser confocal imaging methods to track the motion of cells. Breast cancer cells were seeded on CNT sponge and labeled with a long-term cell-tracking agent. The motion of cells was recorded 48 hours after cell seeding. Time-lapse of cells was recorded using a confocal microscope every two minutes for an hour. The plugin of TrackMate in ImageJ was employed to obtain the statistics of cell motion. Statistical results of cell movement are summarized in FIG. 5a. The final displacement of the CTCs on CNT sponge is significantly reduced compared to those on the glass slides. To further verify the binding strength, 0.25% trypsin medium was used to disassociate the captured CTCs from the substrates (both CNT sponge and glass-f slide) for 10 minutes. FIG. 5b shows the residue cells on both CNT sponge and glass-f slide after the trypsinization. While no cell exists on the glass-f slides, most of the cells remained on the CNT sponge, verifying the strong binding affinity between the CNT sponge and CTCs cannot be damaged by trypsin.

The metabolic activity of CTCs on the CNT sponge provides important information of whether the CNT sponge is beneficial to the growth of tumor cells regarding the primary hypothesis of this method. We have developed a technique based on bioorthogonal click chemistry to visualize the newly synthesized ECM (collagen and glycoprotein molecules) by cells. The newly synthesized proteins were marked with copper-free fluorescent dye (DBCO MB488) and the cells were tracked with long-term live cell tracker (CMTPX). As shown in FIG. 5c, the synthesis of ECM of tumor cells is drastically promoted on the CNT sponge comparing to those on the glass slides. The newly synthesized ECM were further digested with a proteinase (papain), and the fluorescence intensity of the digested solution was obtained to quantify the ECM synthesis. Collagen and glycoproteins synthesis are both promoted in the CTCs captured by 3D CNT sponge (FIG. 5d), indicating that the CTCs can fabricate a favorable microenvironment to survive and invade into the CNT sponge. Synthesis of new ECM of CTCs could serve as extra evidence for tumor cell identification. As normal blood cells do not carry the task of ECM fabrication in animal bodies, the aggressive ECM synthesis could serve as a piece of additional evidence that the captured cells are tumor cells instead of the blood cells.

### CONCLUSION AND REMARKS

To sum up, in order to advance the application of CTC-related diagnostic techniques for the metastasis disease, we designed a CTC capture chip using a 3-dimensional CNT bulk material (3D CNT sponge) that is independent on the tumor-specific markers and uses only a small amount of peripheral blood. The feasibility, efficiency, stability, and accuracy of this CTC detection tool have been systematically investigated by both tumor-cell-spiked blood samples and clinical samples. 20-30% of the CTCs can be quickly captured in merely 15 minutes, and the value yields to 70-80-90% in 60 minutes. The viability and phenotype of captured CTCs can be preserved for further pathological analysis. The fast and reliable capture of CTCs by the CNT sponge material is achieved by providing ideal microenvironments in which the CTCs can attach and progress. Moreover, the CNT sponges can stimulate the synthesis of ECM by CTCs, which not only validates our primary hypothesis but also provides additional evidence to identify the malignancy of the captured cells. This 3D CNT-sponge-based CTC capture chip represents an ideal candidate for the next generation marker-free CTC isolation devices.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method of capturing circulating tumor cells (CTCs) from a subject without using a cancer biomarker, comprising contacting a test sample with a carbon nanotube (CNT) sponge for no more than 60 minutes, wherein the test sample comprises cells from no more than 2 ml peripheral blood from a subject after removal of plasma and lysis of erythrocytes, wherein the cells in the test sample comprise CTCs from the subject, wherein the CNT sponge is free of an agent specific for a cancer biomarker, whereby the CTCs are captured by the CNT sponge.

2. The method of claim 1, wherein the CTCs do not have the cancer biomarker.

3. The method of claim 1, wherein the cancer biomarker is specific to tumor cells.

4. The method of claim 3, wherein the subject has the tumor.

5. The method of claim 3, wherein the tumor is selected from the group consisting of breast cancer, lung cancer and colorectal cancer.

6. The method of claim 1, wherein the cancer biomarker is selected from the group consisting of EpCAM, cytokeratins, CD45 and HER2.

7. The method of claim 1, wherein the test sample comprises 200-1,000 CTCs per ml of the peripheral blood from the subject.

8. The method of claim 1, comprising contacting the test sample with the CNT sponge for no more than 30 minutes.

9. The method of claim 1, comprising contacting the test sample with the CNT sponge for no more than 15 minutes.

10. The method of claim 1, wherein at least 20% of the CTCs in the test sample are captured by the CNT sponge.

11. The method of claim 1, wherein at least one of the captured CTCs remains viable after 7 days in a cell culture.

12. The method of claim 1, further comprising detaching the captured CTCs from the CNT sponge.

13. The method of claim 1, further comprising incubating the captured CTCs in a culture medium.

14. The method of claim 1, further comprising characterizing the captured CTCs.

15. The method of claim 2, wherein the cancer biomarker is specific to tumor cells.

16. The method of claim 4, wherein the tumor is selected from the group consisting of breast cancer, lung cancer and colorectal cancer.

17. The method of claim 2, wherein the cancer biomarker is selected from the group consisting of EpCAM, cytokeratins, CD45 and HER2.

18. The method of claim 3, wherein the cancer biomarker is selected from the group consisting of EpCAM, cytokeratins, CD45 and HER2.

19. The method of claim 4, wherein the cancer biomarker is selected from the group consisting of EpCAM, cytokeratins, CD45 and HER2.

20. The method of claim 5, wherein the cancer biomarker is selected from the group consisting of EpCAM, cytokeratins, CD45 and HER2.
